(19) United States
(12) Patent Application Publication
(10) Pub. No.: US 2013/0095505 A1
(43) Pub. Date: Apr. 18, 2013

(54) DETECTION OF LIPID OXIDISING ABZYMES IN SAMPLES

(71) Applicant: Ivan PETYAEV, Cambridge (GB)

(72) Inventor: Ivan PETYAEV, Cambridge (GB)

(73) Assignee: Cambridge Theranostics Limited, Cambridge (GB)

(21) Appl. No.: 13/439,927

(22) Filed: Apr. 5, 2012

Related U.S. Application Data
(63) Continuation of application No. 11/885,047, filed on Aug. 24, 2007, now abandoned.

(30) Foreign Application Priority Data
Feb. 25, 2005 (GB) 0503940.9

(51) Int. Cl. G01N 33/68

(52) U.S. Cl. CPC G01N 33/6854
USPC 435/7.36; 435/7.4

(57) ABSTRACT

This invention relates to the finding that lipid oxidising abzymes damage Chlamydia antigens in a sample and the extent of damage provides a measure of the level or activity of the abzymes in the sample. Lipid oxidising abzymes may be measured or detected, for example, by abrogating or abolishing abzyme mediated lipid oxidation activity in a sample, and determining the binding of antibodies in the sample to a Chlamydia antigen relative to controls. Such methods may be useful in the assessment of cardiovascular conditions.

This invention relates to the detection of lipid oxidising abzymes in samples of blood or serum. This is useful, for example, in assessing an individual for cardiovascular conditions.

Lipid oxidation is one of the main processes leading to the conversion of circulating lipoproteins into highly atherogenic factors [Goto Y. (1982) supra, Halliwell B., and J. M. C. Gutteridge, (1989) supra, Schultz D., and Harrison D. G. (2000) supra]. The principal cause of lipid oxidation is catalytic antibodies known as ‘abzymes’ (see WO03/017992, WO03/019196 and WO03/019198). Abzymes are a key pathogenic factor in the development of atherosclerosis and are an important diagnostic marker for atherosclerosis-related conditions as well as being a target for therapeutic intervention.

Current abzyme assays are based on the measurement of the oxidation of lipids by the abzymes. The amount of lipid oxidation is, for example, determined from the level of lipid oxidation product malondialdehyde (MDA). MDA levels can be conveniently measured spectrophotometrically, since a coloured product forms when malondialdehyde reacts with thiobarbituric acid [Draper, H. H. et al Free Radic. Biol. Med. (1993) 15, 353].

However, abzyme assays based on lipid oxidation are slow, because the development of lipid oxidation products generally requires 8-12 hours. Lipid oxidation assays also require the use of toxic reagents, such as trichloroacetic acid, and large volumes of patient serum (typically 2-3 ml).

The present inventor has recognised that abzymes damage Chlamydia antigens and this damage can be used to measured abzyme activity in simple, rapid assays using conventional immunoassay formats.

An aspect of the invention provides a method of measuring abzyme levels in a sample comprising;
abrogating or abolishing abzyme mediated lipid oxidation activity in the sample, and
determining the binding of antibodies in the sample to a Chlamydia antigen.

An increase in binding in the sample relative to untreated controls is indicative of the presence of abzymes in the sample. The amount of the increase is indicative of the level of abzymes in the sample. The absence of any increase in binding in the sample relative to controls is indicative of the absence of abzymes from the sample.

The binding of antibodies in samples treated to abolish abzyme mediated lipid oxidation activity and in controls (i.e. untreated samples) may be determined simultaneously or sequentially.

In some embodiments, an initial sample taken from an individual may be divided or aliquoted into two or more separate samples, at least one of which is treated to abrogate or abolish abzyme mediated lipid oxidation and at least one of which remains untreated as a control. In other embodiments, two or more identical samples may be obtained from the individual, at least one of which is treated to abrogate abzyme mediated lipid oxidation and at least one of which remains untreated as a control.

The presence of abzymes in the serum of an individual may be indicative of the individual having or suffering from an atherosclerotic disorder or may be indicative of the susceptibility or risk of the individual suffering from such a disorder in the future. The amount, level or activity of abzymes may be indicative of the severity or level of risk of the disorder i.e. an increase in the amount and/or activity of antibody is indicative of increased severity or risk of disorder.

A method of assessing an individual for an atherosclerotic disorder may comprise;
abrogating or abolishing abzyme mediated lipid oxidation activity in a sample obtained from the individual; and,
determining the binding of antibodies in the sample to a Chlamydia antigen.

The increase in binding in the treated sample relative to untreated sample is indicative of the level of abzymes in the sample. The presence, severity or susceptibility of the individual to an atherosclerotic disorder may be assessed from the level of abzymes in the sample.

These methods may be useful, for example, in determining the optimal therapeutic treatment for an individual. For example, an individual having anti-lipid abzymes indicative of an atherosclerotic condition may be subjected to therapeutic treatment to alleviate the condition or its symptoms. The level or amount of anti-lipid abzymes may be indicative of the severity of the condition and may be used to determine whether or not a particular therapeutic course is appropriate.

A sample is preferably a sample which comprises plasma or serum from the individual, for example a blood, serum or plasma sample. Methods for obtaining, storing and preparing suitable samples from an individual are well known in the medical practice. A test sample of serum may be obtained, for example, by extracting blood from an individual and isolating the serum from the extracted blood. Suitable extraction methods include centrifugation to separate serum and plasma from cellular material.

A Chlamydia antigen may be any immunogen or immunogenic component of a Chlamydia cell i.e. a molecule from Chlamydia which evokes or is capable of evoking an immune response in a mammal against the Chlamydia cell, for example a molecule on the surface of a Chlamydia cell, or a mimetic or functional analogue of such a component. In other words, the Chlamydia antigen is a component of a Chlamydia cell that is capable of specifically binding to antibodies raised against the Chlamydia cell. Suitable Chlamydia antigens for use in the present methods include isolated, purified, cloned or synthesised antigens of Chlamydia, fragments or epitopes of such antigens, groups of such antigens, fraction(s) of Chlamydia cell homogenate, whole Chlamydia cells, and combinations of any of these.

Anti-idiotypic antibodies which imitate active Chlamydia epitopes or fragments of these anti-idiotypic antibodies, may also be suitable for use as Chlamydia antigens in the present methods.

Suitable methods for preparing Chlamydia antigens are well known in the art. For example, Chlamydia antigens may be isolated and/or purified by techniques such as HPLC.

In some preferred embodiments, the Chlamydia antigen may be on the surface of a Chlamydia cell and methods described herein may comprise determining the binding of antibodies in treated and untreated sample to the Chlamydial cell.

A Chlamydial cell may be a cell from a species belonging to the Chlamydia psittaci group. The Chlamydia psittaci group includes Chlamydia psittaci and Chlamydia pneumoniae. In some embodiments, the Chlamydial cell may be an ovine Chlamydia psittaci cell. Suitable preparations of live ovine Chlamydia psittaci in a lyophilised form are available commercially (Intervet).

The binding of antibodies in a sample to a Chlamydia antigen may be determined by any appropriate means or assay format. Tagging with individual reporter molecules is one possibility. For example, a second antibody which binds to antibodies in the sample, or a Chlamydia cell or antigen may be tagged with a reporter molecule. The reporter molecules may directly or indirectly generate detectable, preferably measurable, signals. Where required, linkage of reporter molecules may be direct or indirect, covalent, e.g. via a peptide bond, or non-covalent. Linkage via a peptide bond may be as a result of recombinant expression of a gene fusion encoding binding molecule (e.g. antibody) and reporter molecule.

Reporters include fluorochromes such as fluorescein, rhodamine, phycoerythrin and Texas Red, chromogenic dyes such as diaminobenzidine, macromolecular colloidal particles or particulate material such as latex beads that are coloured, magnetic or paramagnetic, and biologically or chemically active agents that can directly or indirectly cause detectable signals to be visually observed, electronically detected or otherwise recorded.

Biologically or chemically active agents include enzymes which catalyse reactions that develop or change colour or cause changes in electrical properties. Agents may be molecularly excitable, such that electronic transitions between energy states result in characteristic spectral absorptions or emissions. They may include chemical entities used in conjunction with biosensors. Biotin/avidin or biotin/streptavidin and alkaline phosphatase detection systems may be employed. Further examples include horseradish peroxidase and chemiluminescence. Any such method may be used to determine the binding of the antibody to Chlamydia antigen.

The signals generated by individual antibody-reporter conjugates may be used to derive quantifiable absolute or relative data of the relevant antibody binding in samples (normal and test).

Methods of the invention may be carried out in any convenient format. Immunological assays are well-known in the art and many suitable formats are available and may be employed to carry out the present methods, for example ELISA, Western blotting, microimmunofluorescence (MIF), Biacore®, (Biacore, Upsala, Sweden), immunoprecipitation or immuno-turbidimetry, agglutination, for example erythrocyte-, latex- or other polymer-based agglutination, immunohistochemistry, immunoelectrophoresis, antibody-based affinity chromatography and IDEIA® (Boots-Celltech) and other red-ox amplifying diagnostic systems.

In some preferred embodiments, a sandwich assay format may be employed. For example, sandwich assay may employ a capture antibody that binds anti-Chlamydia antibodies in the sample and a labelled Chlamydia antigen or cell that detects the presence of anti-Chlamydia antibodies bound to the capture antibody. Alternatively, a sandwich assay may employ a capture Chlamydia antigen or cell and a labelled antibody which detects the presence of anti-Chlamydia antibodies bound to the antigen. A capture antibody, Chlamydia antigen or Chlamydia cell may be immobilised, for example, by attachment to an insoluble support or solid surface. The support may be in particulate or solid form and may include a plate, a test tube, beads, a ball, a filter or a membrane. Methods for fixing antibodies to insoluble supports are known to those skilled in the art. A non-immobilised component of an assay (i.e. a component which is free in solution) such as an antibody, Chlamydia antigen or Chlamydia cell may comprise a detectable label as described above. For example, the antibody may be labeled with a fluorophore such as FITC or rhodamine, a radioisotope, or a non-isotopic labelling reagent such as biotin or digoxigenin; components containing biotin may be detected using “detection reagents” such as avidin conjugated to any desirable label such as a fluorochrome.

The mode of determining binding is not a feature of the present invention and those skilled in the art are able to choose a suitable mode according to their preference and general knowledge.

The sample may be treated to abrogate abzyme mediated lipid oxidation using any convenient physical or a chemical treatment which abolishes or substantially reduces abzyme activity but which has no effect or substantially no effect on the binding of abzymes to Chlamydia antigens.

In some embodiments, the sample may be physically treated to inactivate abzyme mediated lipid oxidation.

For example, the sample may be heated. The sample may be heated in accordance with any temperature regimen that inactivates lipid oxidation activity but does not affect binding properties of specific antibodies.

A suitable temperature regimen may include heating the sample to at least 37° C., at least 56° C. or at least 70° C. The sample may be heating for a sufficient time to inactivate or substantially inactivate abzyme mediated lipid oxidation without affecting the binding of abzymes to antigen. For example the sample may be heated for at least 1 minute, at least 5 minutes, at least 15 minutes, at least 45 minutes, at least 60 minutes, at least 8 hours, at least 12 hours, or at least 24 hours.

In some embodiments, the sample may be heated to 70° C. for at least 1, at least 2, at least 3, at least 5, or at least 10 minutes; heated to 56° C. for at least 15, at least 20, at least 30, at least 45, or at least 60 minutes; or heated to 37° C. for at least 8 hours, at least 12 hours, at least 24 hours, or at least 48 hours.

Other physical treatments may be used to inactivate lipid oxidation activity without affecting the binding properties of specific antibodies.

The sample may be subjected to repetitive freeze-thaw cycles, for example two or more cycles of freezing followed by thawing.

The sample may be subjected to prolonged storage, for example at least 4 days at 0° C. to 4° C., at least 2 or at least 3 months at −10° or at least 4 or at least 6 months at −20° C.

The sample may be subjected to high-energy ultrasound, microwave, UV, gamma radiation or any other electro magnetic waves.

The suitability of a treatment or regimen for use in the present methods may be determined by measuring the lipid oxidation and Chlamydia-binding activity of the sample after treatment, as described herein. A suitable treatment or regimen for use in the present methods inactivates abzyme mediated lipid oxidation but does not affect the binding properties of anti-Chlamydia antibodies

In other embodiments, the sample may be chemically treated to inactivate abzyme mediated lipid oxidation. For example, the sample may be treated with one or more abzyme inactivating agents.

Inactivating agents may include low pH antioxidants (i.e. inhibits oxidation reactions at pH5.5), hydroxyl radical scavengers, ‘electron trappers’ such as crown ethers and steroids, ‘electron cushions’ such as polyvinyl-based polymers, ‘electron sinks’, such as ubiquinones and Q₈, copper chelators and calcium chelators.

Suitable inactivating agents may include ascorbic acid, acetyl salicylic acid, sodium azide, catechins, including catechin gallate, DMSO, azithromycin, haemoglobin, telithromycin ketek, or derivatives, analogues and salts of any of these.

In other embodiments, an inactivating agent may be a bacterial cell, for example a cell from probiotic bacteria such as lactobacilli, or a product of such a cell.

The efficacy of a treatment in inactivating abzymes may be determined by determining lipid oxidation activity of a sample of abzymes, for example IgG obtained from a patient atheroma, before and after treatment. Any convenient method of determining lipid oxidation may be used. Many methods for determining lipid oxidation are known in the art and may be used to determine the reduction or abrogation of lipid oxidation activity in a sample. Suitable methods are, for example, described in CRC Handbook of Methods for Oxygen Radical Research, CRC Press, Boca Raton, Fla. (1985), Oxygen Radicals in Biological Systems. Methods in Enzymology, v. 186, Academic Press, London (1990); Oxygen Radicals in Biological Systems. Methods in Enzymology, v. 234, Academic Press, San Diego, New York, Boston, London (1994); and Free Radicals. A practical approach. IRL Press, Oxford, New York, Tokyo (1996) In preferred embodiments, oxidation is determined by determining the production (i.e. the presence or amount) of a lipid oxidation product, which may include aldehydes such as malondialdehyde (MDA), (lipid) peroxides, diene conjugates or hydrocarbon gases.

The sample from the individual may be treated to inhibit or reduce complement activity. In some embodiments, the sample may be treated with a complement inhibitor. Inhibitors of complement activity are well known in the art and include, for example, Ca²⁺ chelators such as EGTA or EDTA, thymidine kinase inhibitors, including catechins such as epigallocatechin gallate (EGCG), polysaccarides such as zymosan, peptidyl molecules such as CD46, CD55, CD59, pexelizumab, eculizumab, compstatin, Cobra venom, antibodies against Clq and other components or intermediates of complement cascade, and fragments of these antibodies, and compounds which imitate the functions and properties of the complement cascade.

In other embodiments, the sample may be treated with a procedure or regimen that inhibits complement activity. Suitable procedures include heating the sample, for example to 56° C. for 30 minutes, or 70° C. for 2-5 minutes, or other temperature regimen that inactivates complement. Other physical procedures, such as ultrasound shock, irradiation and/or laser treatment, may also be used.

The methods of determining abzyme activity that are described herein may also be useful in screening for compounds which inhibit abzyme activity. Such compounds may be useful in the treatment of atherosclerotic conditions.

Another aspect of the invention provides a method of screening for an abzyme inhibitor comprising;
determining the binding of antibodies in a sample to a Chlamydia antigen,
treating the sample with a test compound, and;
determining the binding of antibodies in the treated sample to a Chlamydia antigen.

An increase in binding to a Chlamydia antigen after said treatment is indicative that the compound is an abzyme inhibitor.

The sample is preferably a sample comprising abzymes. A suitable sample may be obtained from the individual having an atherosclerotic disorder and may, for example, be a serum sample or sample from an atheroma or atherosclerotic lesion. The sample may be enriched for IgG, for example, by binding with protein A, as described below. The presence of abzymes in the sample may be confirmed using lipid oxidation assays which are known in the art.

An abzyme inhibitor identified by these methods may be useful in the treatment of a atherosclerotic disorder, including a cardiovascular disorder such as atherosclerosis, ischaemic (coronary) heart disease, myocardial ischaemia (angina), myocardial infarction, aneurismal disease, atheromatous peripheral vascular disease, aortoiliac disease, chronic and critical lower limb ischaemia, visceral ischaemia, renal artery disease, cerebrovascular disease, stroke, atherosclerotic retinopathy, thrombosis and aberrant blood clotting, and hypertension. Such conditions may be medical or veterinary conditions.

A suitable test compound may be a small chemical entity, peptide, antibody molecule or other molecule whose effect on abzyme activity is to be determined. Suitable test compounds may be selected from compound collections and designed compounds, for example using combinatorial chemistry as described below. Particular suitable test compounds include metal chelators, for example copper or calcium chelators, antioxidants, in particular low pH anti-oxidants (i.e. compounds which inhibit oxidation reactions at pH 5.5), hydroxyl radical scavengers, ‘electron trappers’ such as crown ethers or steroids, ‘electron cushions’ such as polyvinyl-based polymers and ‘electron sinks’, such as ubiquinones and Q₈.

Suitable test compounds may include analogues, salts and derivatives of sodium azide, catechins, including catechin gallate, DMSO, azithromycin, haemoglobin and telithromycin, which have been identified as abzyme inhibitors using the present methods and fractions, extracts and derivatives of bacterial cells, in particular, cultures of probiotic bacteria such as lactobacilli.

Combinatorial library technology (Schultz, JS (1996) Biotechnol. Prog. 12:729-743) provides an efficient way of testing a potentially vast number of different substances for ability to modulate the activity of abzymes. Prior to or as well as being screened as described above, test compounds may be screened for ability to bind with the abzyme. This may be used as a coarse screen prior to testing a compound for actual ability to modulate abzyme activity.

The amount of test compound which may be added to an assay of the invention will normally be determined by trial and error depending upon the type of compound used. Typically, from about 0.01 to 100 nM concentrations of putative inhibitor compound may be used, for example from 0.1 to 10 nM.

Compounds which may be used may be natural or synthetic chemical compounds used in drug screening programmes. Extracts of plants which contain several characterised or uncharacterised components may be used or extracts, fractions or components of probiotic bacteria, such as lactobacilli.

Other candidate inhibitor compounds may be based on modelling the 3-dimensional structure of the abzyme and/or the lipid antigen to which it binds and using rational drug design to provide potential inhibitor compounds with particular molecular shape, size and charge characteristics.

A screening method described herein may further comprise determining the lipid oxidation activity of an abzyme in the presence of the test compound.

Lipid oxidation activity, including lipid peroxidation activity, may be determined by determining the oxidation of host lipid (i.e. lipid from the sample), lipid from a foreign antigen such as a Chlamydia cell, or lipid from another source, which may for example be added as part of an assay method. The accumulation of oxidation products or by-products, such as co-oxidised coupled reporter molecules, may be measured or the disappearance or consumption of substrates such as non-modified lipids or co-substrates such as oxygen. Many methods for determining lipid peroxidation are known in the art and are suitable for use in accordance with the present invention. Suitable methods are, for example, described in CRC Handbook of Methods for Oxygen Radical Research, CRC Press, Boca Raton, Fla. (1985), Oxygen Radicals in Biological Systems. Methods in Enzymology, v. 186, Academic Press, London (1990); Oxygen Radicals in Biological Systems. Methods in Enzymology, v. 234, Academic Press, San Diego, New York, Boston, London (1994); and, Free Radicals. A practical approach. IRL Press, Oxford, New York, Tokyo (1996)

In preferred embodiments, oxidation is determined by determining the production (i.e. the presence or amount) of lipid oxidation products, include aldehydes such as malondialdehyde (MDA), (lipid) peroxides, diene conjugates or hydrocarbon gases. Lipid oxidation products may be determined by any suitable method. For example, lipid peroxidation products may be determined using HPLC (Brown, R. K., and Kelly, F. J In: Free Radicals. A practical approach. IRL Press, Oxford, New York, Tokyo (1996), 119-131), UV spectroscopy (Kinter, M. Quantitative analysis of 4-hydroxy-2-nonenal. Ibid., 133-145), or gas chromatography-mass spectrometry (Morrow, J. D., and Roberts, L. J. F₂-Isoprostanes: prostaglandin-like products of lipid peroxidation. Ibid. 147-157). The production of malondialdehyde (MDA), for example, may be determined, following reaction with 2-thiobarbituric acid (conveniently at 1 mM), by measuring absorbance at an appropriate wavelength, for example 525 nm.

An agent identified using one or more primary screens (e.g. in a cell-free system) as having ability to inhibit abzyme activity may be assessed further using one or more secondary screens. A secondary screen may involve testing for abzyme activity in the vascular system or for a biological function of an abzyme, for example, in an animal model. Suitable biological functions which may be assessed in a secondary screen include reduction in size or number of atherosclerotic lesions, or a reduction in other symptoms or effects of an atherosclerotic disorder, such as blood pressure.

Methods of the present invention may include identifying a test compound as an agent that inhibits abzyme activity.

Examples of compounds identified as abzyme inhibitors using the present methods include ascorbic acid, acetyl salicylic acid, sodium azide, (+) catechin gallate, DMS, haemoglobin, telithromycin-ketek, lactobacillus cells and other agents as set out in Table 3.

The identified compound may be isolated or purified and/or synthesised or manufactured.

Optionally, a compound identified as abzyme inhibitor as described herein may be modified to optimise activity or provide other beneficial characteristics such as increased half-life or reduced side effects upon administration to an individual. Techniques and strategies for the modification of lead compounds are well known in the art.

An abzyme inhibitor identified described herein may be formulated into a composition, such as a medicament, pharmaceutical composition or drug, with a pharmaceutically acceptable excipient as described below. Such a composition may be administered to an individual.

The present invention encompasses a compound identified using an assay method described above as abzyme inhibitor, a pharmaceutical or veterinary composition, medicament, drug or other composition comprising such a compound, a method comprising administration of such a composition to a patient, e.g. for treatment (which may include preventative treatment) of atherosclerotic conditions, use of such a compound in manufacture of a composition for administration, e.g. for treatment of an atherosclerotic condition, and a method of making a pharmaceutical or veterinary composition comprising admixing such a compound with a pharmaceutically acceptable excipient, vehicle or carrier, and optionally other ingredients.

Whether it is a polypeptide, peptide, nucleic acid molecule, small molecule or other pharmaceutically useful compound that is to be given to an individual, administration is preferably in a “prophylactically effective amount” or a “therapeutically effective amount” (as the case may be, although prophylaxis may be considered therapy), this being sufficient to show benefit to the individual. The actual amount administered, and rate and time-course of administration, will depend on the nature and severity of what is being treated. Prescription of treatment, e.g. decisions on dosage etc, is within the responsibility of general practitioners and other medical doctors.

A composition may be administered alone or in combination with other treatments, either simultaneously or sequentially dependent upon the condition to be treated.

Pharmaceutical compositions according to the present invention, and for use in accordance with the present invention, may include, in addition to active ingredient, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous or intravenous.

Pharmaceutical compositions for oral administration may be in tablet, capsule, powder or liquid form. A tablet may include a solid carrier such as gelatin or an adjuvant. Liquid pharmaceutical compositions generally include a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, or Lactated Ringer's Injection. Preservatives, stabilisers, buffers, antioxidants and/or other additives may be included, as required.

Those of skill in the art may vary the precise format of assay methods of the invention using routine skill and knowledge.

Various further aspects and embodiments of the present invention will be apparent to those skilled in the art in view of the present disclosure. All documents mentioned in this specification are incorporated herein by reference in their entirety.

The invention encompasses each and every combination and sub-combination of the features that are described above.

Certain aspects and embodiments of the invention will now be illustrated by way of example and with reference to the figures and tables described below.

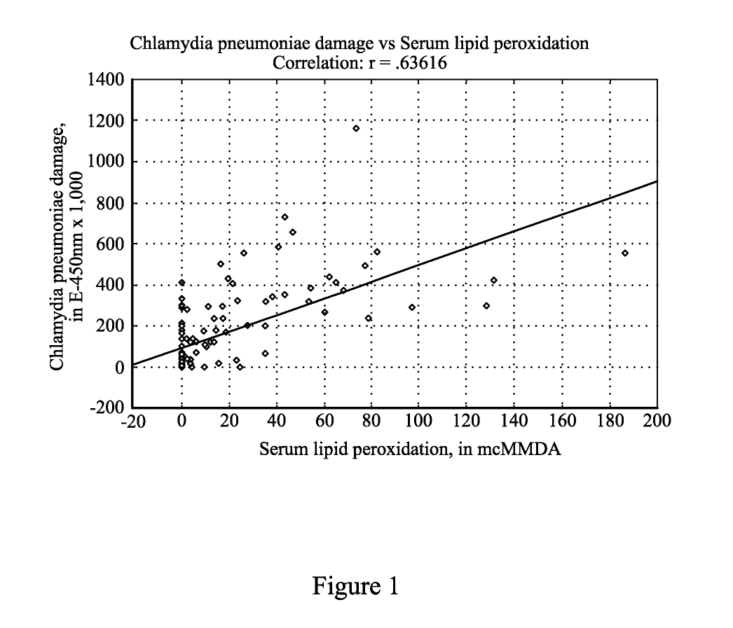

FIG. 1 shows a comparison of lipid oxidation and Chlamydia antigen damage assays to measure the activity of anti-Chlamydia abzymes,

Table 1 shows a comparison of the measurement of the activity of anti-Chlamydia abzymes either via their ability to oxidise serum lipids or via their ability to damage Chlamydia antigen, ELISA assay

Table 2 shows a comparison of the measurement of the activity of anti-Chlamydia abzymes either via their ability to oxidise serum lipids or via their ability to damage Chlamydia antigen, MIF assay.

Table 3 shows the effect of different factors on the ability of the abzymes to cause lipid peroxidation and damage the Chlamydia pneumoniae antigen(s).

### EXAMPLES

### Materials and Methods

### Preparation of Samples

Antibodies were extracted from advanced atherosclerotic lesions of human aorta retrieved from two male patients of 53 and 64 years old, during bypass surgery of an abdominal aortal stenosis at the Centre of Cardio-Vascular Surgery of the Medical University of Rostov-na-Donu, Russian Federation. After recovery these samples were immediately put in 30% w/v solution of NaCl and stored at 0-4° C. for 1 month prior to examination. In the control experiments it was shown that during this period, the activities of such enzymes as trypsin, catalase, superoxide dismutase, glutathione peroxidase, creatine kinase and lactate dehydrogenase, together with a level of immunoglobulin (IgG) fragmentation and the degree of lipid peroxidation did not significantly change.

The pieces of aorta (approximately 200-400 mg wet weight) were cut into pieces of approximately 10 mg each, placed in 5.0 ml of PBS with 1% non-ionic detergent Igepal CA-630 and homogenised by a mechanical homogeniser (Ultra-Turrax) at full-power with a 15 mm probe three times for 3 seconds each with 20 second cooling intervals. After homogenisation the insoluble components were separated by centrifugation at 5000 g for 10 minutes and supernatants were used for analysis

The supernatant was treated with protein A attached to cross-linked 4% beaded agarose at 37° C. for 30 minutes. The immunoglobulin fraction attached to the beads was then spun down at 5000 g for 10 minutes and the supernatant decanted. In order to remove any lipoproteins attached to the sedimented immunoglobulins, the samples were re-suspended with 10% of Igepal CA-630. They were then centrifuged at 5000 g for 10 minutes and the supernatant was decanted.

To remove the detergent three subsequent washings were performed in the excess of the phosphate buffer with centrifugation under the same regime. The removal of lipoprotein from the immunoglobulin fraction was confirmed by the absence of cholesterol in this fraction.

### Inactivation of Abzymes

For physical inactivation, the same abzyme sample was split into two aliquots. One aliquot was heated in a water bath for 30 minutes at 56° C. The other was untreated. Following treatment of the first aliquot, both samples were tested in the same fashion.

For chemical inactivation, a diluent solution was divided into two portions. In one portion, an abzyme inhibitor was added. The following abzyme inhibitors were used DMSO, 0.1-10%; sodium azide, 10⁻⁵-10⁻³M; catechins, 10⁻⁶-10⁻³M; ketek, 10⁻⁶-10⁻³M; lactobacilli culture, 1 μM-1 mM; ascorbic acid, 10⁻⁴-10⁻³M; acetyl salicylic acid, 10⁻⁴-10⁻³M.

The serum sample was split in two aliquots and one aliquot was diluted by solution containing the abzyme inhibitor, the second aliquot was diluted by the control solution. The aliquots were then tested in the same fashion.

### ELISA Based Abzyme Assay

ELISA assays were performed using Medac materials and reagents, which were used in accordance with the manufacturers instructions.

Briefly, serum samples from patients were treated as described above. 50 μl of sample diluent was pipetted into microtitre well Al as blank, and 50 μl of the negative control, Positive Control and the diluted patients' samples were pipetted into other microtitre wells. The microplate wells were incubated for 60 min (±5 min) at 37° C. (±1° C.) in a humid chamber and then washed three times with 200 μl wash buffer per well. 50 μl of Conjugate was then added to each well and the microplate wells incubated again for 60 min (±5 min) at 37° C. (±1° C.) in a humid chamber and then washed. 50 μl of TME-Substrate, was added to each well and the microplate wells incubated for 30 min (±2 min) at 37° C. (±1° C.) in a humid chamber. The reaction was stopped by adding 100 μl of Stop Solution, to each well.

Photometric reading was performed at 450 nm (ref. 620-650 nm) within 15 min after adding the Stop Solution.

To calculate the results, the OD value of the blank (well Al) was subtracted from all other OD values. Preferably, the OD value of the blank was <0.150, the mean OD value of the Negative Control was <0.100 and the OD value of the Positive Control was >0.800. Cut-off=mean OD value of the Negative Control+0.380. Grey zone=Cut-off±10%

### Microimmunofluorescence (MIF) on Lysed Chlamydia

Slides with antigens of Chlamydia trachomatis, C. psittaci, and C. pneumoniae were prepared by applying purified elementary bodies of these bacteria. Sera were diluted to a titer of 1:1024 in phosphate-buffered saline (PBS) and incubated for 30 min at 37° C. After washing in PBS, anti-human IgG, IgA, IgM conjugates were added to the samples. After 30 mins of incubation at 37° C. and being washed in PBS, the slide was covered with a cover slip with mounting medium.

A fluorescent microscope was used for the reading of the slides. A positive reaction is represented by a “starry sky” appearance: fluorescent green spots on a slightly red background.

Two independent experts evaluated all samples.

### Analysis of ELISA and MIF Results

If there was a difference less than 10% (or no increase in titers in MIF) of the signal between treated and non-treated samples the conclusion was that abzyme activity was negative.

If there was a difference between 10 and 15% (or an increase in one titer in MIF) of the signal between treated and non-treated samples the conclusion was that the activity of the abzymes was unclear, or in the “grey” zone.

If there were a difference of more than 15% (or an increase in two titers or more in MIF) of the signal between treated and non-treated samples the conclusion was that the abzyme activity was positive.

### Electron Microscopy on Lysed Chlamydia

Bacteria cells were fixed for 1 hour in 2.5% solution of glutaraldehyde, made in 0.2 M cacodylic buffer pH 7.2, after that in chrome-osmium solution for another hour.

After that samples were dehydrated in a gradient increase of ethanol and absolute acetone and imbedded in Eponate 12T14-Araldite 502. Ultra-thin slides were made by using Ultracut Reichert-Jung, stained by 1% water solution of uranyl acetate and lead citrate.

Slides were examined and photographed using an electron microscope JEM 100C×(with magnification of)×5300-53000 times.

### SDS-PAGE

Polyacrylamide gel electrophoresis was performed using various commercially available systems, in accordance with the manufacturer's instructions. For example, the method described in DPO 033/02; Issue 1.0 “Protein electrophoresis using NOVEX™ system (SDS-PAGE)” was used with the following reagents: NuPAGE™ Bis-Tris 4-12% precast gels (Invitrogen NP0321 batch #2063076) (15 well); NuPAGE™ Bis-Tris 4-12% precast gels (Invitrogen NP0321 batch #2072272) (10 well); NuPAGE™LDS sample buffer 4×(Invitrogen NP0007 batch #300277); NuPAGE™ Sample reducing agent x10 (Invitrogen NP0004 batch #300505) NuPAGE™ MOPS SOS running buffer x20 (Invitrogen NP0001 batch #300704); SeeBlue™ pre stained markers (Invitrogen LC5625 batch #see11214).

### Determination of Peroxidation of Lipids

Lipid peroxidation was assessed as a level of MDA concentration, which was measured by spectrophotometric methods [Draper, H. H. et al Free Radio. Biol. Med. (1993) 15, 353]. Briefly, the level of abzymes in a sample was determined as follows: Samples of sera were diluted 1:1 by 0.05M acetate buffer pH 4.0 to make the final pH of these samples between 5.6-5.8. 994 μl of the diluted serum was mixed with 10 μl of the commercial live ovine Chlamydia vaccine (Intervet). Samples were incubated overnight (12-16 hours) at 37° C. 250 μl of 40% trichloroacetic acid and 250 μl of 1 mM 2-thiobarbituric acid was added to each sample. All samples were placed in a water bath and boiled for 30 minutes. Samples were cooled down and centrifuged at 3,000 g for 10 minutes. The supernatants were collected and their absorption measured at λ525 nm to determine the concentration of malondialdehydes (MDA), which are products of lipid peroxidation.

### Results

### Effect of Abzymes on Chlamydia

An abzyme-enriched fraction of atheroma IgG was prepared as described above. The effect of the abzyme-enriched fraction of atheroma IgG on Chlamydia pneumoniae elementary and recticulocyte bodies was determined by MIF and electron microscopy.

The abzyme-enriched fraction was observed to lyse Chlamydia pneumoniae cells in a microimmunofluorescence assay (MIF). Electron microscopy showed that the abzyme-enriched fraction lysed both Chlamydia pneumoniae elementary and reticular bodies.

### Analysis of Abzyme-Enriched Fraction of Atheroma IgG

The abzyme-enriched fraction of atheroma IgG was analysed by SDS-PAGE along with an IgG fraction purified from serum.

Electrophoresis analysis showed that the abzyme-enriched fraction contained only IgG and no any other detectable proteins.

### Abzyme Assays

ELISA and MIF assays for Chlamydia antigen damage as described above were used to measure the activity of anti-Chlamydia abzymes in patient serum samples, in comparison with lipid oxidation assays. The results are set out in tables 1 and 2, respectively and summarised in FIG. 1.

The results of the ELISA (in E₄₅₀ₙₘ×1,000) and MIF (Ab titer) assays were observed to correlate with the results of lipid peroxidation assays.

### Abzyme Inhibition

A range of treatments were tested for ability to inactivate abzymes by ELISA as described above and the lipid oxidation assay for ability to inhibit abzyme activity. The results are set out in table 3.

Ascorbic acid, acetyl salicylic acid, sodium azide, EDTA, EGTA, catechin gallate, DMSO, haemoglobin, telithromycin ketek, and lactobacilli were all observed to inhibit abzyme activity.

Compounds which inactivate abzymes may be useful in methods of for determining abzyme levels or may be useful in therapy, for example the treatment of atherosclerotic or cardiovascular conditions.

TABLE 1

| Abzyme-negative sera (n = 33), | | Abzyme positive sera (n = 33) | |
|---|---|---|---|
| measurement of the activity | | measurement of the activity | |
| based on: | | based on: | |
| | Chlamydia | | Chlamydia |
| | pneumoniae | | pneumoniae |
| serum | antigen damage, | serum | antigen damage, |
| peroxidation, | in ELISA | peroxidation, | in ELISA |
| in μM MDA | E_{450 nm} × 1,000 | in μM MDA | E_{450 nm} × 1,000 |
| 0 | 139 | 186 | 550 |
| 6 | 271 | 128 | 296 |
| 4 | 136 | 37 | 283 |
| 9 | 307 | 35 | 207 |
| 3 | 15 | 131 | 424 |
| 0 | 0 | 54 | 390 |
| 0 | 31 | 82 | 564 |
| 0 | 0 | 77 | 494 |
| 0 | 8 | 47 | 660 |
| 0 | 16 | 35 | 322 |
| 0 | 63 | 27.5 | 206 |
| 0 | 43 | 38 | 345 |
| 0 | 0 | 43.5 | 732 |
| 3 | 124 | 41 | 584 |
| 0 | 61 | 65 | 413 |
| 0 | 100 | 60 | 267 |
| 0 | 166 | 26 | 555 |
| 9 | 278 | 19 | 432 |
| 0 | 8 | 78.5 | 241 |
| 0 | 28 | 62 | 443 |
| 0 | 0 | 21 | 409 |
| 0 | 4 | 12 | 124 |
| 0 | 11 | 23 | 324 |
| 0 | 11 | 18 | 172 |
| 0 | 14 | 17 | 298 |
| 0 | 16 | 97.5 | 293 |
| 0 | 26 | 13 | 237 |
| 0 | 204 | 43 | 356 |
| 0 | 6 | 53 | 320 |
| 0 | 0 | 74 | 1158 |
| 10 | 99 | 68 | 376 |
| 0 | 0 | 24 | 376 |
| 0 | 0 | 17 | 238 |
| 1.3 ± 0.025 | 58 ± 13.7 | 52.8 ± 6.17 | 388 ± 31.4 |
| | | p < 0.001 | p < 0.001 |

TABLE 2

Abzyme activity in terms of the loss of the
antibody ability to damage Chlamydia
pneumoniae antigen

| | | |
|---|---|---|
| | MIF titers before | MIF titers after |
| Patients | abzyme inactivation | abzyme inactivation |

Abzyme (+) sera, tested by lipid oxidation MDA assay

| | | |
|---|---|---|
| P577 | 0 | 1/128 |
| OAG | 0 | 1/64 |
| YIO | 0 | 1/64 |
| IVM | 0 | 1/64 |
| IMK | 0-1/16 | 1/64 |
| P580 | 0 | 1/64 |
| P573 | 0 | 1/64 |
| P571 | 0 | 1/32 |
| AFP | 0 | 1/16-1/32 |
| VAM | 0 | 1/16 |
| P572 | 0 | 0 |

Abzyme (−) sera, tested by lipid oxidation MDA assay

| | | |
|---|---|---|
| P585 | 0 | 0 |
| AIS | 0 | 0 |
| GPM | 0 | 0 |
| P567 | 0 | 0 |
| INK | 0 | 0-1/16 |
| SII | 0 | 0-1/16 |
| NEC | 0 | 0 |
| JON | 0 | 0-1/16 |
| KAT | 0 | 0 |
| JIM | 0 | 0 |

TABLE 3

| | Inhibition of abzymes ability to cause: | |
|---|---|---|
| | Serum lipid | Damage of Chlamydia |
| Factors affecting | peroxidation, in | pneumoniae antigen, |
| abzyme activity | MDA assay | in ELISA |

Physical procedures

| | | |
|---|---|---|
| Repetitive freezing thawing | Positive | Positive |
| Heating at 56° C. for 30 min | Positive | Positive |

Drugs, reagents or food products

| | | |
|---|---|---|
| 1. Acetyl salicylic acid | Positive | Positive |
| 2. Ascorbic acid | Positive | Positive |
| 3. EDTA | Positive | Positive |
| 4. EGTA | n/a | Positive |
| 5. Sodium cyanide | Negative | Negative |
| 6. Sodium azide | Positive | Positive |
| 7. (+) Catechin gallate | Positive | Positive |
| 8. β-Carotene | Negative | Negative |
| 9. (+) α-Tocopherol | Negative | Negative |
| 10. (+) y-Tocopherol | Negative | Negative |
| 11. Benzoic acid | Negative | Negative |
| 12. DMSO | Positive | Positive |
| 13. D-Mannitol | Negative | Negative |
| 14. PMS | Negative | Negative |
| 15. Haemoglobin | Positive | Positive |
| 16. Telithromycin, Ketek | Positive | Positive |
| 17. Tetracycline | Negative | Negative |
| 18. Lactobacilli culture | Positive | Positive |
| 19. Lycopene | Negative | Negative |

* antibody-antigen reaction was blocked by lowered pH after addition of these acid compounds.

1. A method of measuring abzyme levels in a sample comprising;
abolishing abzyme mediated lipid oxidation in said sample, and
determining the binding of antibodies in the sample to a Chlamydia antigen,
wherein an increase in binding in the treated sample relative to controls is indicative of the presence or level of abzymes in the sample.

2. A method according to claim 1 wherein the sample is a blood, serum or plasma sample obtained from an individual.

3. A method according to claim 2 wherein the presence of abzymes in the sample is indicative of an atherosclerotic condition.

4. A method according to claim 1 wherein the sample is physically treated to abrogate abzyme mediated lipid oxidation.

5. A method according to claim 4 wherein the sample is heated.

6. A method according to claim 5 wherein the sample is heated to at least 37° C. for at least 5 minutes.

7. A method according to claim 6 wherein the sample is heated to at least 56° C. for at least 30 mins.

8. A method according to claim 4 wherein the sample is exposed to two or more freeze thaw cycles.

9. A method according to claim 4 wherein the sample is maintained at 0° C. to 4° C. for at least 4 days.

10. A method according to claim 1 wherein the sample is chemically treated to abrogate abzyme mediated lipid oxidation.

11. A method according to claim 10 wherein the sample is treated with one or more inactivating agents.

12. A method according to claim 11 wherein the inactivating agent is a hydroxyl radical scavenger, low pH anti-oxidant, electron trapper, cushion or sink.

13. A method according to claim 11 wherein the inactivating agent is a hydroxyl radical scavenger

14. A method according to claim 11 wherein the inactivating agent is selected from the group consisting of acetyl salicylic acid, ascorbic acid, EDTA, EGTA, (+) catechin gallate, sodium azide, DMSO, haemoglobin telithromycin ketek and analogues or derivatives thereof.

15. A method according to claim 11 wherein the inactivating agent is a bacterial cell

16. A method according to claim 15 wherein the inactivating agent is a lactobacillus cell.

17. A method according to claim 1 wherein the amount of abzyme mediated lipid oxidation in the sample is determined after said treatment.

18. A method according to claim 1 wherein the Chlamydia antigen is on the surface of a Chlamydia cell.

19. A method according to claim 1 wherein the binding of the antibody to the Chlamydia antigen is determined using a second antibody.

20. A method according to claim 19 wherein the second antibody binds to IgG.

21. A method according to claim 19 wherein a first member of the group consisting of the second antibody and the Chlamydia antigen or cell is labelled.

22. A method according to claim 21 wherein a second member of the group consisting of the second antibody and the Chlamydia antigen or cell is immobilised.

23. A method of screening for an abzyme inhibitor comprising;
determining the binding of antibodies in a sample to a Chlamydia antigen,
treating the sample with a test compound and;
determining the binding of antibodies in the treated sample to a Chlamydia antigen,
an increase the binding of the treated sample relative to the untreated sample being indicative that the compound is an abzyme inhibitor.

24. A method according to claim 23 wherein the abzyme inhibitor is for the treatment of an atherosclerotic disorder.

25. A method according to claim 23 wherein the sample comprises lipid oxidising anti-Chlamydia abzymes.

26. A method according to claim 25 wherein the sample is from an individual having an atherosclerotic disorder.

27. A method according to claim 26 wherein the sample is a serum or atheroma sample.

28. A method according to claim 23 wherein the sample is an IgG enriched sample.

29. A method according to claim 23 wherein the inactivating agent is a low pH antioxidant.

30. A method according to claim 23 wherein the inactivating agent is a hydroxyl radical scavenger.

31. A method according to claim 23 comprising determining the lipid oxidation activity of an abzyme in the presence of the test compound.

32. A method according to claim 23 comprising identifying the compound as an abzyme inhibitor.
